(19) United States
(12) Patent Application Publication
(10) Pub. No.: US 2022/0152330 A1
(43) Pub. Date: May 19, 2022

(54) Anesthetized Catheter, Indication System and Methods of Administering, Removing and Testing Substances

(71) Applicant: Blaine Cameron, Plantation, FL (US)

(72) Inventor: Blaine Cameron, Plantation, FL (US)

(21) Appl. No.: 17/529,154

(22) Filed: Jan. 3, 2022

Related U.S. Application Data
(63) Continuation of application No. 63/114,614, filed on Nov. 17, 2020.

(51) Int. Cl. A61M 16/04; A61M 16/01

(52) U.S. Cl. CPC A61M 16/0463; A61M 2205/44; A61M 2205/3553; A61M 16/01

(57) ABSTRACT

An anesthetized catheter that includes the ability to administer substances and remove substances via the catheter orifice as well as pericatheter with the option of using wireless technology, while also having an indication system.

### CROSS-REFERENCE TO RELATED APPLICATIONS

This is a continuation application of Blaine Cameron, Anesthetized Catheter, Indication System and Methods of Administering, Removing and Testing Substances, U.S. Appl. No. 63/114,614, filed Nov. 17, 2020 and incorporated herein by reference in its entirety.

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### STATEMENT REGARDING FEDERALLY SPONSORED RESEARCH OR DEVELOPMENT

Not Applicable

### THE NAMES OF PARTIES TO A JOINT RESEARCH AGREEMENT

Not Applicable

### INCORPORATION-BY-REFERENCE OF MATERIAL SUBMITTED ON A COMPACT DISC

Not Applicable

### BACKGROUND OF THE INVENTION

### Field of the Invention

### Description of the Related Art

In medical practice, various invasive catheters are inserted into the body for various purposes. An endotracheal intubation involves the insertion of a tube into the patient's airway. The problem is that this is a very densely innervated area and is very stimulating and painful. Other examples of catheters in use today include, foley catheter inserted into the urethra, nasogastric catheter inserted into the nares and pharynx and into the stomach other catheters such as the Dubhoff are advanced to the small intestine. Chest tubes are inserted into the lung between the ribs into the pleural space.

Pain is sensed through the afferent pain pathway. Multiple cortical and subcortical structures are involved in the experience of pain. Tissue damage due to surgery initiates the release of local inflammatory mediators (e.g. Bradykinin, substance P, prostaglandins, potassium, histamine, and serotonin). The primary goal of analgesia is patient comfort. Secondary goals include attenuation of adverse physiologic responses to pain (e.g. Hypermetabolism, increased oxygen consumption, hypercoagulability, and alterations in immune function. Prevention of development of chronic pain syndromes. Control of anxiety and agitation. Systemic treatment can result in side effects from e. G. Opioids. Depressed consciousness, depression of respiratory drive, hallucination and delirium, hypotension, histamine release, peripheral vasodilation, nausea and vomiting Ileus, Urinary retention, Pruritus, increased intracranial pressure, effects on immune system tolerance, withdrawal and hyperalgesia.

Current practitioners attempt to treat this pain and discomfort by administering systemic pain medications such as narcotics. And anesthetic drugs. It is therefore necessary to sedate intubated patients post operatively e. G. Post cardiac surgery. Or ICU patients. The problem is these medications are sedating, depress cardiorespiratory system and therefore have the exact opposite from the desired outcome, an awake alert patient who is breathing adequately and whose vital signs are stable. Many new sedating drugs have been introduced into the ICU setting for example that have less respiratory depression. The attendant problems of this system include but are not limited to pain, delirium, dyspnea, anxiety, paralysis, cost, excess sedation, PTSD, and withdrawal.

This systemic treatment retards current anesthesia/critical care/medical goals and objectives e. G. Fast track cardiac surgery pathways. The aim is to facilitate extubating of patients within 1-6 hours after cardiac surgery. Most centers consider fast track extubating up to 8-10 hours post-operation.

These systemically administered medication also in addition to causing sedation, also cause delirium. Pain is the most common memory patients have of their ICU stay. Agitation can precipitate accidental removal of catheters and intra-vascular catheters used for monitoring or administration of life sustaining medications. Consequently, sedatives and analgesics are among the most administered drugs in the ICUs.

Pain, over sedation, and delirium are issues that are distressing to patients and associated with increased morbidity and mortality. Prospective studies confirm that the majority of patients who are treated in ICUs have pain. Short-term consequences of untreated pain include higher energy expenditure and immunomodulation. Longer-term, untreated pain increases the risk of post-traumatic stress disorder. Practitioners have thus Providers cannot currently be abstemious.

There is therefore a need for a treatment specifically designed for the task of treating pain, thus reducing agitation and not causing or exacerbating delirium.

### SUMMARY OF THE INVENTION

An example embodiment of the present invention is directed to an anesthetized catheter which includes the ability to administer substances and remove substances via the catheter orifice as well as pericatheter with the option of using wireless technology, while also having an indication system.

One object is to provide an anesthetized catheter.

Another object is to provide an anesthetized catheter that provides local anesthesia to the area that the catheter is situated.

Another object is to provide an anesthetized catheter that can provide a drug delivery system.

Another object is to provide an anesthetized catheter that can provide patient controlled analgesia via, wireless technology (bluetooth/wifi). To reduce transduction of pain and the release of substance P etc.

Another object is to provide an anesthetized catheter that improved compliance, patient satisfaction and facilitate expedient hospital discharge. e.g. post-operative cardiac “fast tracking.”

Another object is to provide an anesthetized catheter that facilitates the placement, maintenance and discontinuation or removal of painful catheters including but not limited to endotracheal tubes, foley catheters, suprapubic catheters, nephrostomy tubes, orogastric tubes, nasogastric tubes, chest tubes, jejunal tubes, drainage tubes, rectal tubes.

Another object is to provide an anesthetized catheter that facilitates diagnostic specimen removal. e.g. COVID 19 testing from endotracheal tubes, chest tubes and nasogastric tubes.

Another object is to provide an anesthetized catheter that it facilitates therapeutic drug administration and analgesic and or anesthetic drug administration that can be patient controlled.

Another object is to provide an anesthetized catheter that has an indicator catheter that provides feedback as to correct catheter placement and has diagnostic capabilities.

There has thus been outlined, rather broadly, some of the features of the cameron catheter in order that the detailed description thereof may be better understood, and in order that the present contribution to the art may be better appreciated. There are additional features of the cameron catheter that will be described hereinafter and that will form the subject matter of the claims appended hereto. In this respect, before explaining at least one embodiment of the cameron catheter in detail, it is to be understood that the cameron catheter is not limited in its application to the details of construction or to the arrangements of the components set forth in the following description or illustrated in the drawings. The cameron catheter is capable of other embodiments and of being practiced and carried out in various ways. Also, it is to be understood that the phraseology and terminology employed herein are for the purpose of the description and should not be regarded as limiting.

Other objects and advantages of the various embodiments of the present invention will become obvious to the reader and it is intended that these objects and advantages are within the scope of the present invention. To the accomplishment of the above and related objects, this invention may be embodied in the form illustrated in the accompanying drawings, attention being called to the fact, however, that the drawings are illustrative only, and that changes may be made in the specific construction illustrated and described within the scope of this application.

### BRIEF DESCRIPTION OF THE DRAWINGS

Features and advantages of the present invention will become apparent to those skilled in the art from the following description with reference to the drawings, in which:

Example embodiments will become more fully understood from the detailed description given herein below and the accompanying drawings, wherein like elements are represented by like reference characters, which are given by way of illustration only and thus are not limitative of the example embodiments herein.

FIG. 1: shows an anesthetized catheter ETT of the present invention. A catheter with delivery system, wireless capability and indicator in the catheter lumen. A tubular catheter. A balloon about at least a portion of the catheter. The balloon being inflatable from a collapsed configuration, in addition to the balloon fixing the portion of the catheter. A drug dispensation catheter balloon configured to contact the mucosa of the endotracheal tube, urethra, surgical incision, pleura, esophagus, etc. This catheter is not designed for intravascular use as it can cause intravascular rupture, occlusion and/or systemic toxicity, seizure, cardiac arrythmia and death.

Option of distal port for spray injection of e. G. Lidocaine to anesthetize the airway for intubation.

Intra-luminal carbon dioxide indicator. Pilot balloon. Nylon outer sleeve port. Balloon with A. Woven nylon sleeve or alternatively PCA drug dispensation.

B. Coated collapsible balloon or.

C. Microneedles or.

D. Micro-adhesive with releasable drug per PCA.

FIG. 2 is an upper perspective view of the present invention. PCA device remote to control PCA. Alternatively, the device can be controlled by an APP on phone such as android, apple or other smart phone device. This can reduce substance P.

FIG. 3: FIG. 3 is an end view of the present invention. Cross sectional view of catheter with outer woven nylon sleeve. Can see on cross section inner catheter lumen as well as outer diameter catheter lumen e. G. Where local anesthetic would be injected and seep through the outer wall to anesthetize the surrounding tissue.

FIG. 4: FIG. 4 is a right-side view of a urethral catheter embodiment of the present invention. Example of urethral catheter with indicator dipstick for diagnosis, HCG testing. Woven outer sleeve. Collapsible balloon. Alternative also incorporate distal port for injection of lidocaine jelly for insertion.

FIG. 5: FIG. 5 is a top view an anesthetized catheter NGT of the present invention. Nasogastric tube alternatively PEG tube or Dubhoff tube. Woven outer nylon sleeve which can be customized to contact the painful areas in the oropharynx only.

FIG. 6: FIG. 6 is a bottom view of an anesthetized catheter surgical drain of the present invention. Surgical drain, JP drain version of an anesthetized catheter. Customizable woven nylon sleeve to function at the skin and subcutaneous tissues.

FIG. 7: FIG. 7 is an exploded upper perspective view chest tube of the present invention. Chest tube version of an anesthetized catheter with adjustable woven nylon sleeve which can be customized for the patient by the thoracic surgeon to be in contact with the skin and subcutaneous tissue to anesthetize the painful areas of the chest tube insertion. This can prevent the release of substance P etc. Improve respiratory effort and reduce atelectasis.

### DETAILED DESCRIPTION OF THE INVENTION

### A. Overview

Turning now descriptively to the drawings, in which similar reference characters denote similar elements throughout the several views, the figures illustrate an example embodiment comprising the ability to administer substances and remove substances via the catheter orifice as well as pericatheter with the option of using wireless technology. while also having an indication system.

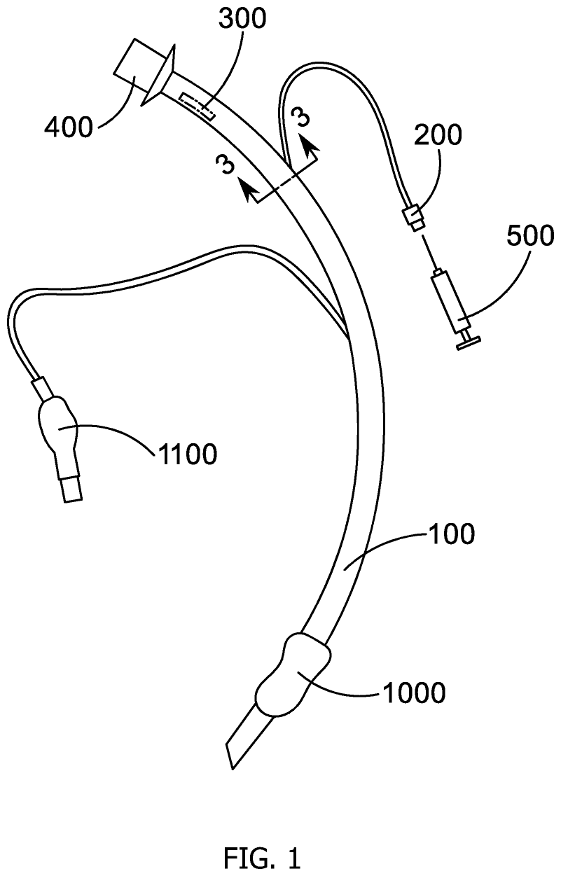

Shown in FIG. 1 are: catheter main tube 100; nylon sleeve port 200; indicator 300; connector 400; manual injection source 500; endotracheal tube cuff 1000; and, pilot balloon 1100.

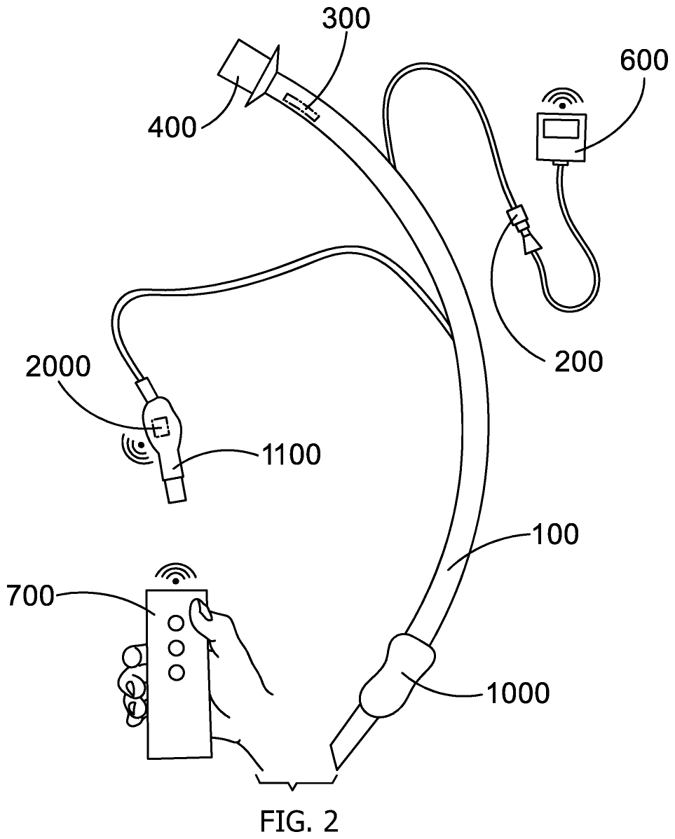

Shown in FIG. 2 are: catheter main tube 100; nylon sleeve port 200; indicator 300; connector 400; wireless injection source 600; wireless user control 700; endotracheal tube 1000; pilot balloon 1100; and, pilot balloon wireless receiver 2000.

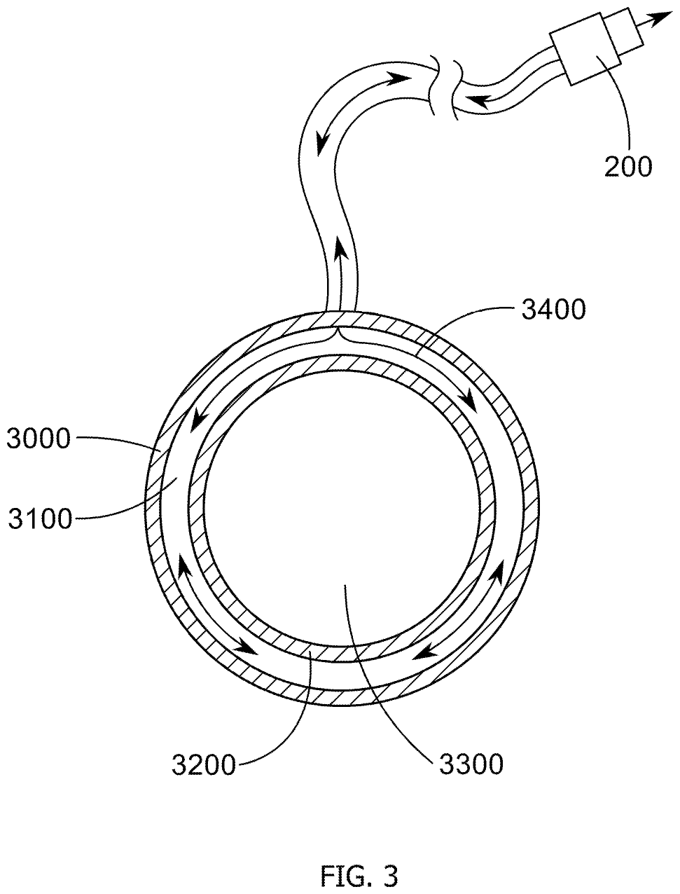

Shown in FIG. 3 are: nylon sleeve port 200; outer woven nylon sleeve 3000; outer catheter lumen 3100; inner woven nylon sleeve 3200; inner catheter lumen 3300; and injection/extraction fluid flow 3400.

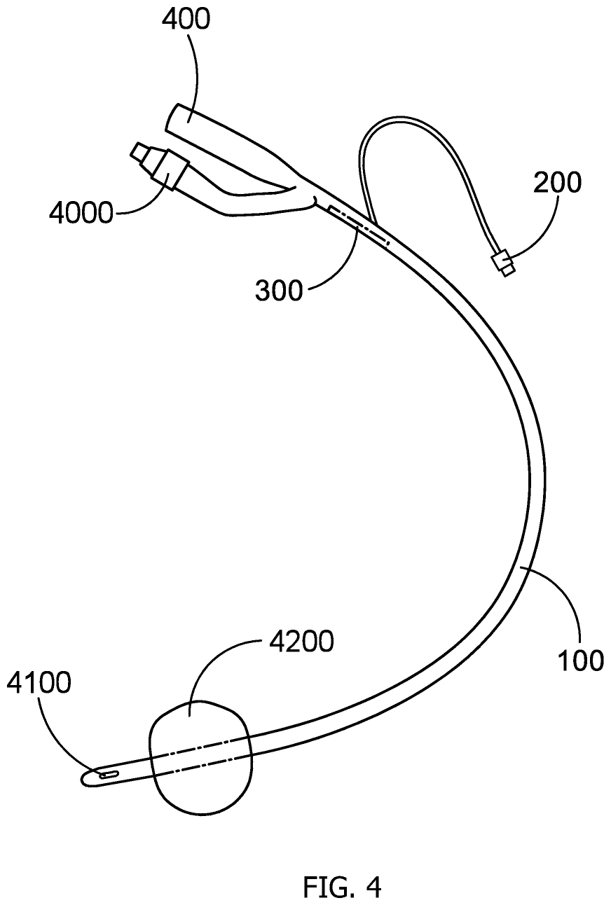

Shown in FIG. 4 are: catheter main tube 100; nylon sleeve port 200; indicator 300; connector 400; bladder balloon inflation point 4000; urine intake 4100; and, bladder balloon 4200.

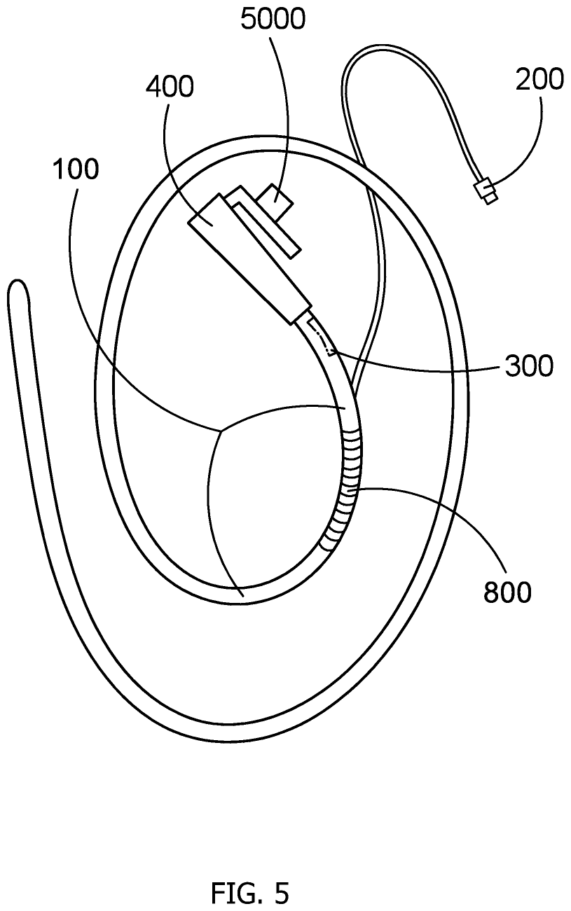

Shown in FIG. 5 are: catheter main tube 100; nylon sleeve port 200; indicator 300; connector 400; machine customizable portion 800; and, nasogastric tube plug 5000.

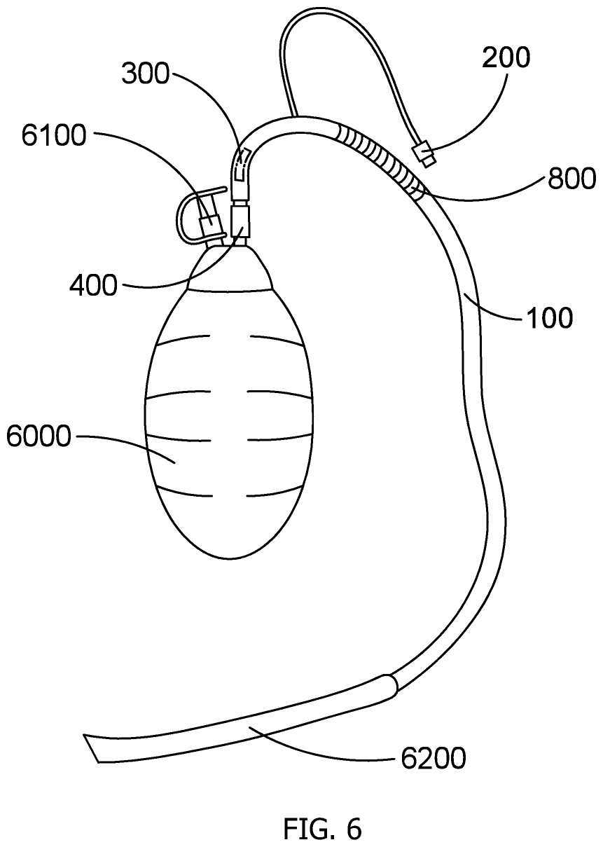

Shown in FIG. 6 are: catheter main tube 100; nylon sleeve port 200; indicator 300; connector 400; machine customizable portion 800; surgical drain bulb 6000; stopper 6100; and drainage end 6200.

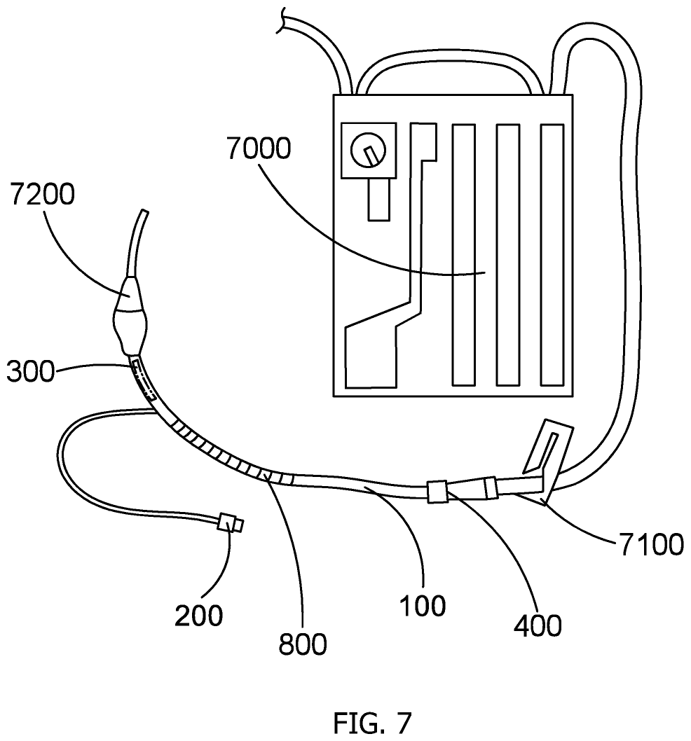

Shown in FIG. 7 are: catheter main tube 100; nylon sleeve port 200; indicator 300; connector 400; machine customizable portion 800; chest tube drainage management system 7000; patient tube clamp 7100; and, clearance apparatus patient insertion end 7200.

### B. Cameron Catheter Substance Administration

The anesthetized catheter achieves substance delivery, transmucosal/cutaneous/dermal/ureteral/luminal lung, colon, nasal, oral, pharyngeal and others. Balloon device with dual function of maintaining catheter placement and allowing direct contact with tissue can provide substance administration, dilution and withdrawal etc. The balloon is part of the catheter. Dual purpose of maintaining catheter placement/location and agent administration or substance withdrawal. Balloon can be radio opaque or translucent.

Unlike other tubes and catheters that can administer substances from its distal or proximal ends. These catheters can do so around the perimeter of the catheter. Surrounding the catheter and thus have the ability to anesthetize any tissue the anesthetized catheter may come in contact or close proximity and thus stimulate or cause pain.

Alternatively the anesthetized catheter can be covered in woven nylon sleeve which administers the substance such as local anesthetics.

Presently very few practitioners administer local anesthesia pre catheter insertion. E. G pre intubation lidocaine injection, lidocaine gel pre foley insertion and more frequently subcutaneous injection of local anesthetic for chest tube placement. This is limited to one time and thus usually 1 hour of anesthesia. And once the catheter is in place cannot be repeated. For example, once endotracheal tube is in place it is difficult to administer local anesthesia around the endotracheal (ETT) tube. Same issue with Foley etc. The anesthetized catheter allows for repeated administration/drainage of substances via catheter or on the outer surface of the catheter. This allows for repeated administration, drainage, anesthesia, chemotherapy, antibiotic, antiviral etc.

Custom substance delivery port. Machine tool used by medical provider to customize placement of delivery/drainage ports before placement of catheter. Not an intravascular device and thus has a balloon which can be inflated and remain inflated without vascular damage and rupture. Drug administration is local and while it can be converted to systemic is not main intention as e. G. Fleming's device. Also has an outer layer to catheter for potential transdermal/transmucosal/etc. Administration.

### C. Cameron Catheter Wireless Technology Patient Controlled Analgesia

The anesthetized catheter has wireless Patient controlled Analgesia. While Patient—controlled analgesia (PCA) has been utilized since 1971 it has not in the last approximately 50 years been used for local anesthesia in the trachea, nasogastric, urethra, lung pleura or rectaly via peri catheter approach and not using wireless technology. PCA has been proven more effective at pain control than non-patient injections and results in higher patient satisfaction. Either by sonification of liposomes or logarithmic programs with non-corrodible materials. Receiver in the balloon. External remote. Dispensation mechanism outside of patient. Alternative external sonification device.

Blue tooth, Wi-Fi, sonification device, telemetry, radiofrequency, laser, digital, cellular, radio, fiberoptic.

Wireless technology allowing patient-controlled administration PCA, provider controlled administration. Telemedicine. May use Wi-Fi, Bluetooth. May incorporate sonification of liposomes or by logarithmic programs with remote administration control. Receiver in balloon or at end of tube.

External remote, Receiver contained in pilot balloon. Dispensation mechanism outside of patient. Deliver at port. Alternatively, external sonification device. Liposomes in outer tube coating. Bluetooth, Wi-Fi, sonification device, telemetry, radiofrequency, laser, digital, cellular radio, microwave or fiberoptic.

### D. Cameron Catheter Substance Withdrawal Therapeutic and Diagnostic

Ability to remove and test substances. Reagent strip in catheter. Different catheters with different strips. E. G ETT tube with CO2 monitor to confirm endotracheal placement. While this functionality exists in separate equipment it has never been combined. This combination gives functional ability to continuously monitor for ETT extubating. Foley catheter and nephrostomy tube with urine dipstick strip in catheter, (multistix or chemstrip) HCG, (pregnancy testing preoperatively. Biuret solution. Etc.

The anesthetized catheter has the ability to allow for the withdrawal of substances pericatheter. Specifically, on the outer diameter of the catheter in addition to distally or proximally. This allows for example. withdrawal of urine in urinary catheter, fluid from chest tubes, etc.

### E. Cameron Catheter Coated Outer Diameter

Generally, the drug dispensing film is compressed or crimped onto the balloon portion of the catheter so that it does not move longitudinally relative to the balloon portion of the catheter.

The coated outer diameter will not contain any type of corrosive metal. There may be an aspect of mucoadherence. It can be transcellular or paracellular in route depending on whether lipophilic or hydrophilic drugs are used.

### F. Cameron Catheter Microneedles

Alternate microneedles can be source of substance delivery. Can infiltrate local anesthetic and reduce transduction and transmission of pain signals and the release of substance P.

This embodiment is one option for drug delivery of local anesthetic and local anesthetic infiltration to the mucosal wall/peri-catheter placement.

### G. Cameron Catheter Balloon Delivery Device

The anesthetized catheter functions as current catheters such as endotracheal tube, foley catheter, NGT, nephrostomy tube, surgical drain, J tube dubhoff catheter chest tube rectal tube, dual function of balloon as opposed to current balloon which has one function to place catheter this balloon has the additional function of administration or drainage pericatheter using wireless technology and machine tool allows for customization of the catheter.

A balloon about at least a portion of the catheter, the balloon having a first end and a second end and a working length in between, the first end and the second end each including a tapered portion, each tapered portion being attached to the catheter, the balloon being inflatable from a collapsed configuration, wherein the working length and at least a portion of each tapered portion are substantially flattened, to an inflated configuration.

Custom local anesthetics.

Amylocaine, Ambucaine, Articaine, Benzocaine, Benzonatate, Bupivacaine, Butacaine, Butanilicaine, beckercaine, Chloroprocaine, Cinchocaine, Cocaine, cetacaine, Cyclomethycaine, Dibucaine, Diperodon, Dimethocaine, Eucaine, Etidocaine, Hexylcaine, Fomocaine, Fflecainide, Fotocaine, Hydroxyprocaine, Isobucaine, Levobupivicaine, Lidocaine, Mepivicaine, Meprylcaine, Metabutoxycaine, Nitracaine, Orthocaine, Oxetacaine, Oxybuprocaine, Parathoxycaine, Phenacaine, Piperocaine, Piridocaine, Pramocaine, Prilocaine, Primacaine, Procaine, Procainamide, Proparacaine, Propoxycaine, Quiisocaine, Ropivicaine, Trimecaine, Tetracaine, Tolycaine, Tropacocaine.

Menthol, peppermint oil, mint oil, capsazepine, capsaicin, piperazinyl urea, phenylacetylrinvanil, A425619, JYL 1421anandamide, cannabidiol, Lecithin-coated microdroplets of methoxyflurane, drug coated Poly(L-lactide)Palmitoylethanolamide, AMG9810, AMG0347, SB 452533, SB366791SB705498, SB782443, GRC 6211, Imidazole, 5 aminoisoquinoline TRPV1 antagonist.

While the present invention has been described with regards to the preferred embodiment, it is understood that variation to the present invention may be made without departing from the concept of the present invention. For example, the port at the distal end or peritubular may vary, the use of Wi-Fi or Bluetooth may vary without departing from the inventive concept disclosed herein.

### H. Cameron Catheter Customization of Catheter Machine Tool

There is a machine tool device that can be used to customize the catheter before it is inserted. Drug administration is local and not systemic as in P Fleming device and not an intravascular catheter. Also has an outer layer for potential, transmucosal, transdermal etc. Administration. Machine device puncture site can be altered. Sliding mechanism, gear mechanism. Alternate fixed tools. Machine tools for various size catheters.

The machine tool is a customizable device that can alter the location of delivery and thus customize the delivery of substances to the tissue with which it is proximal to.

### I. Cameron Catheter Indication System

The anesthetized catheter has within the inner wall of the transparent catheter a reagent treated strip that indicates the presence of certain substances. For example, ETT has carbon dioxide indicator strip built into the inner wall and visible through the catheter thus confirming placement in the trachea. Urine dipstick reagent in urinary catheter indicating the presence of protein, infection etc. Indicator strip in chest tube, NGT and surgical drain. Etc with reagent treated test indicator strips being specific to the catheter type.

The catheter indication system consists of an indicator strip either embedded in the luminal (translucent) wall of the catheter or in a side port of the catheter. Endo-tracheal tubes will have a litmus paper embedded in the translucent wall of the catheter and will thus indicate the presence of carbon dioxide and thus confirm placement and maintenance of endo-tracheal tube placement. Urethra with “dipstick” embedded in catheter lumen walls to evaluate urine pH, hematuria, proteinuria (kidney function), glycosuria, ketonuria (diabetes) nitrites (infection), leukocytes (infection), bilirubin (liver disease). Similarly, suprapubic catheters. Chest tubes, Surgical drains, NGT/Dubhoff H. pylori dipstick embedded in wall. Guiac testing in rectal catheters. Test for heme in NGT and Dubhoff tubes as well as J tubes.

Thus the catheter can also be a surveillance tool. For example, monitoring and detecting infection in indwelling catheters and suprapubic catheters. By the catheter being a part of the catheter wall, it eliminates dead space and the risk of disconnection in ETT. It reduces the risk of infection and contamination in the current method of obtaining urine from indwelling catheters and supra-pubic catheters.

### J. Connections of Main Elements and Sub-Elements of Invention

The embedded indicator strips can alternatively be contained in a side port. This would not be advantageous in the ETT as this would increase dead space. This can be helpful in the NGT, Dubhoff, urethral catheter, chest tube and also can be used as part of a disposable portion of the inserted intrathecal catheter when inserting an intrathecal pump delivery system and intrathecal needle.

### K. Operation of Preferred Embodiment

By a paradigm shift of treatment of catheter stimulation of pain from systemic medication to local anesthesia the provider can avoid the deleterious effects of systemic treatment. The provider is also able to reduce transduction of pain and substance P. Also, true allergy to amide local anesthetics are extremely rare. Allergic reactions to local anesthetics are usually a reaction to epinephrine, vasovagal syncope, overdose toxicity or attributed to additives such as metabisulfite or methylparaben.

Another way to administer local anesthesia presently in use, is by lidocaine injection pre catheter insertion. This is limited. The anesthetized catheter allows pre injection as well as maintenance injection for the entire duration of the catheter in situ.

The reduction in pain can reduce substance P, reduce other pro inflammatory mediators release due to pain. Additionally, this can reduce the change in C fibres and reduce the incidence of chronic pain because of catheter placement. This reduction in pain also reduces the need for sedation and can improve cardiorespiratory function hastening extubating and discharge.

The anesthetized catheter enables a paradigm shift from general sedation for pain and systemic treatment of catheter related pain to local anesthetic treatment of catheter related pain which is now not currently done with adequate frequency or duration.

Unless otherwise defined, all technical and scientific terms used herein have the same meaning as commonly understood by one of ordinary skill in the art to which this invention belongs. Although methods and materials like or equivalent to those described herein can be used in the practice or testing of the cameron catheter, suitable methods and materials are described above. All publications, patent applications, patents, and other references mentioned herein are incorporated by reference in their entirety to the extent allowed by applicable law and regulations. The cameron catheter may be embodied in other specific forms without departing from the spirit or essential attributes thereof, and it is therefore desired that the present embodiment be considered in all respects as illustrative and not restrictive. Any headings utilized within the description are for convenience only and have no legal or limiting effect.

Having thus described preferred embodiments, it should be apparent to those skilled in the art that certain advantages of the described system have been achieved. It should also be appreciated that various modifications, adaptions, and alternative embodiments thereof may be made within the scope and spirit of the present invention. The invention is further defined by the following claims.

What is claimed is:

1. An anesthetized catheter that includes the ability to administer substances and remove substances via the catheter orifice as well as pericatheter with the option of using wireless technology, while also having an indication system.

2. A method of administering or removing substances via catheter orifice and pericatheter with the option of using wireless technology, while also having an indication system.

3. A system for pericatheter anesthesia described herein.
